(19) United States
(12) Patent Application Publication
(10) Pub. No.: US 2010/0076430 A1
(43) Pub. Date: Mar. 25, 2010

(54) Electrosurgical Instrument Having a Thumb Lever and Related System and Method of Use

(75) Inventor: Paul R. Romero, Loveland, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/236,666

(22) Filed: Sep. 24, 2008

(51) Int. Cl. A61B 18/12

(52) U.S. Cl. 606/51

(57) ABSTRACT

An electrosurgical instrument and system and method for performing electrosurgery therewith is disclosed. The electrosurgical system includes an electrosurgical generator adapted to supply electrosurgical energy to tissue. The electrosurgical system includes an electrosurgical instrument that provides a shaft, such as a laparoscopic shaft, having at the distal end thereof an end effector. The end effector may include without limitation a pair of movable jaws adapted to perform tissue fusion and/or vessel sealing. The electrosurgical instrument includes a movable thumb lever in operable communication with the shaft to enable the surgeon to rotate the shaft in an ergonomic, single-handed manner.

### BACKGROUND

1. Technical Field

The present disclosure relates to an electrosurgical forceps and more particularly, the present disclosure relates to an endoscopic bipolar electrosurgical forceps having a shaft rotatable by the selective actuation of a thumb lever.

2. Background of Related Art

Electrosurgical forceps utilize both mechanical clamping action and electrical energy to affect hemostasis by heating the tissue and blood vessels to coagulate, cauterize and/or seal tissue. Many surgical procedures require cutting and/or ligating large blood vessels and large tissue structures. Due to the inherent spatial considerations of the surgical cavity, surgeons often have difficulty suturing vessels or performing other traditional methods of controlling bleeding, e.g., clamping and/or tying-off transfected blood vessels or tissue. By utilizing an elongated electrosurgical forceps, a surgeon can either cauterize, coagulate/desiccate and/or simply reduce or slow bleeding simply by controlling the intensity, frequency and duration of the electrosurgical energy applied through the jaw members to the tissue. Most small blood vessels, i.e., in the range below two millimeters in diameter, can often be closed using standard electrosurgical instruments and techniques. However, larger vessels can be more difficult to close using these standard techniques.

In order to resolve many of the known issues described above and other issues relevant to cauterization and coagulation, a recently developed technology has been developed by Valleylab, Inc. of Boulder, Colo., a division of Covidien, called vessel or tissue sealing. The process of coagulating vessels is fundamentally different than electrosurgical vessel sealing. For the purposes herein, “coagulation” is defined as a process of desiccating tissue wherein the tissue cells are ruptured and dried. “Vessel sealing” or “tissue sealing” is defined as the process of liquefying the collagen in the tissue so that it reforms into a fused mass with limited demarcation between opposing tissue structures. Coagulation of small vessels is sufficient to permanently close them, while larger vessels and tissue need to be sealed to assure permanent closure.

In order to effectively seal larger vessels (or tissue) two predominant mechanical parameters are accurately controlled—the pressure applied to the vessel (tissue) and the gap distance between the electrodes—both of which are affected by the thickness of the sealed vessel. More particularly, accurate application of pressure is important to oppose the walls of the vessel; to reduce the tissue impedance to a low enough value that allows enough electrosurgical energy through the tissue; to overcome the forces of expansion during tissue heating; and to contribute to the end tissue thickness which is an indication of a good seal. Various force-actuating assemblies have been developed in the past for providing the appropriate closure forces to affect vessel sealing. For example, one such actuating assembly has been developed by Valleylab, Inc. of Boulder, Colo., a division of Covidien, for use with Valleylab's vessel sealing and dividing instrument for sealing large vessels and tissue structures commonly sold under the trademarks LIGASURE™, LIGASURE 5mm™, LIGASURE ATLAS®. The LIGASURE ATLAS® is presently designed to fit through a 10 mm cannula and includes a bi-lateral jaw closure mechanism and is activated by a foot switch. Co-pending U.S. application Ser. Nos. 10/179,863, 10/116,944, 10/460,926, 10/953,757, and 11/595,194, and PCT Application Serial Nos. PCT/US01/01890 and PCT/7201/11340 describe in detail the operating features of the LIGASURE devices and various methods relating thereto. The contents of each of these applications are hereby incorporated by reference herein.

During electrosurgical procedures, such as vessel sealing, the particular characteristics of a patient's anatomy may require the surgeon to employ specific surgical techniques. For example, the angle at which a vessel is to be sealed may be dictated by adjacent anatomical structures, as well as the target vessel itself. Anatomical structures may dictate that a surgeon manipulate an electrosurgical instrument in a precise manner in order, for example, to traverse a path to the surgical site. Such manipulations may include varying the attitude of the instrument jaws in order to achieve the desired operative result.

### SUMMARY

The present disclosure is directed to an electrosurgical instrument having a housing that includes a movable thumb handle disposed thereon that is rotatable about a first axis defined by a driveshaft having a first end and a second end. The driveshaft is operably coupled at a first end thereof to the thumb handle and a second handle thereof to a drive assembly. The electrosurgical instrument in accordance with the present disclosure includes a shaft coupled to the housing and rotatable about a second axis defined longitudinally therethrough and having an end effector disposed at a distal end thereof for performing an electrosurgical procedure. In use, a surgeon may rotate the shaft and end effector by manipulating the thumb handle, for example, in a leftward or rightward direction. Advantageously, an instrument in accordance with the present disclosure allows a surgeon to manipulate the instrument, including effectuating the rotation of the shaft, using a single hand.

The disclosed electrosurgical instrument includes a drive assembly configured to couple the driveshaft and the rotatable shaft, wherein a rotation of the thumb handle and driveshaft is translated into a rotation of the rotatable shaft. In embodiments, the drive assembly includes a driving element operably coupled to a second end of the driveshaft and a driven element operably coupled to a proximal end of the shaft. The driving element and driven element cooperate to translate rotation therebetween. In embodiments, the driving element and/or driven element may be a bevel gear or friction roller configured to cooperate to translate rotational motion therebetween.

Also disclosed is an electrosurgical system that includes an electrosurgical generator configured to generate electrosurgical energy. The electrosurgical generator may be operatively coupled to the presently disclosed electrosurgical instrument for performing electrosurgical procedures, for example without limitation, cutting, blending, coagulating, ablation, and vessel sealing. In embodiments the electrosurgical generator may supply electrosurgical signals in the radiofrequency range, for example without limitation the 200 kHz-3.3 mHz range, and/or the electrosurgical generator may supply electrosurgical signals in the microwave range, for example without limitation the 900 mHz-2.0 gHz range.

A method of performing electrosurgery is disclosed herein which includes the steps of providing an electrosurgical module configured to generate electrosurgical energy; providing the electrosurgical instrument described hereinabove; providing a cable assembly configured to operably couple the electrosurgical module and the electrosurgical instrument; actuating the movable thumb handle to rotate the end effector; and applying electrosurgical energy to tissue. In embodiments, the end effector assembly provides two jaw members movable from a first position in spaced relation relative to one another to at least a second position closer to one another for grasping tissue therebetween. In embodiments, the disclosed method includes the step of positioning the jaw members around tissue therebetween and moving the jaw members from a first position in spaced relation relative to one another to at least a second position closer to one another to grasp tissue therebetween.

### BRIEF DESCRIPTION OF THE DRAWINGS

The above and other aspects, features, and advantages of the present disclosure will become more apparent in light of the following detailed description when taken in conjunction with the accompanying drawings wherein:

FIG. 1 is an oblique view of an electrosurgical system in accordance with the present disclosure;

FIG. 2 is a cutaway view of an exemplary electrosurgical instrument having a thumb lever in accordance with the present disclosure;

FIG. 3A is a perspective view of an exemplary electrosurgical instrument in accordance with the present disclosure having a thumb lever in a center position;

FIG. 3B is a perspective view of an exemplary electrosurgical instrument in accordance with the present disclosure having a thumb lever in a left position;

FIG. 3C is a perspective view of an exemplary electrosurgical instrument in accordance with the present disclosure having a thumb lever in a right position;

FIG. 4A is a perspective view of an electrosurgical instrument in accordance with the present disclosure shown with an end effector in a first position to grasp and seal a tubular vessel or bundle through a cannula;

FIG. 4B is a perspective view of an electrosurgical instrument in accordance with the present disclosure shown with an end effector in a second position to grasp and seal a tubular vessel or bundle through a cannula;

FIG. 5A is a view of thumb lever having a thumb saddle in accordance with the present disclosure; and

FIG. 5B is a view of thumb lever having a thumb paddle in accordance with the present disclosure.

### DETAILED DESCRIPTION

Particular embodiments of the present disclosure will be described hereinbelow with reference to the accompanying drawings; however, it is to be understood that the disclosed embodiments are merely exemplary of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In the drawings and in the descriptions which follow, the term “proximal,” as is traditional, shall refer to the end of the instrument which is closer to the user, while the term “distal” shall refer to the end which is farther from the user. Relative terms, such as “left”, “right”, “clockwise”, and “counterclockwise” shall be construed from the perspective of the user, i.e., from a proximal viewpoint facing distally.

The present disclosure includes an electrosurgical apparatus that is adapted to connect to an electrosurgical generator that includes a control module configured for electrosurgical procedures.

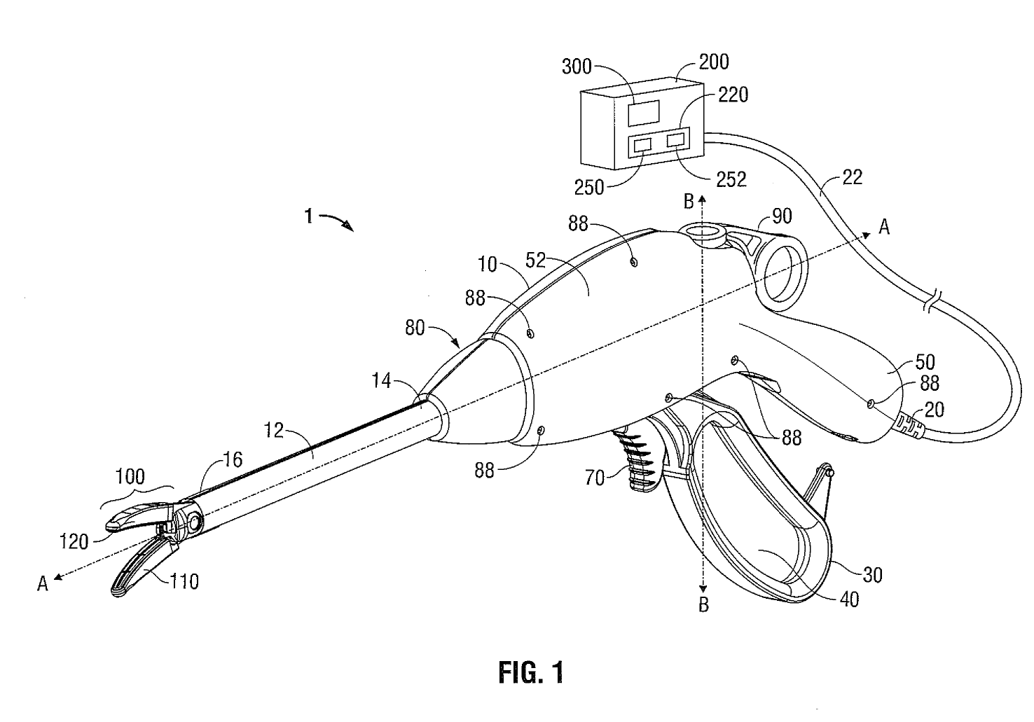

With reference to FIG. 1, an illustrative embodiment of an electrosurgical system 1 is shown. Electrosurgical system 1 includes a generator 200 that is configured to operatively and selectively couple to electrosurgical instrument 10 for performing an electrosurgical procedure. It is to be understood that an electrosurgical procedure may include without limitation seating, cutting, coagulating, desiccating, and fulgurating tissue, all of which may employ RF energy. Electrosurgical instrument 10 may be a bipolar forceps. Generator 200 may be configured for monopolar and/or bipolar modes of operation.

With particular respect to the prior disclosure, generator 200 includes a control module 300 that is configured and/or programmed to control the operation of generation of 200, including without limitation the intensity, duration, and waveshape of the generated electrosurgical energy, and/or accepting input, such as without limitation user input and sensor input. Generator 200 generates electrosurgical energy, which may be RF (radio frequency), microwave, ultrasound, infrared, ultraviolet, laser, thermal energy or other electrosurgical energy. An electrosurgical module 220 generates RF energy and includes a power supply 250 for generating energy and an output stage 252 which modulates the energy that is provided to the delivery device(s), such as an end effector 100, for delivery of the modulated energy to a patient. Power supply 250 may be a high voltage DC or AC power supply for producing electrosurgical current, where control signals generated by the system 300 adjust parameters of the voltage and current output, such as magnitude and frequency. The output stage 252 may modulate the output energy (e.g., via a waveform generator) based on signals generated by control module 300 to adjust waveform parameters, e.g., waveform shape, pulse width, duty cycle, crest factor, and/or repetition rate. Control module 300 may be coupled to the generator module 220 by connections that may include wired and/or wireless connections for providing the control signals to the generator module 220.

As shown in FIG. 1, electrosurgical instrument 10 also includes an electrosurgical cable 22 which connects the electrosurgical instrument 10 to the generator 200. Cable 22 is internally divided into cable leads (not explicitly shown) which are designed to transmit electrical potentials through their respective feed paths through the electrosurgical instrument 10 to the end effector 100. It is contemplated that generators such as those sold by Valleylab, a division of Covidien, located in Boulder, Colo. may be used as a source of electrosurgical energy, e.g., Ligasure™ Generator, FORCE EZ™ Electrosurgical Generator, FORCE FX™ Electrosurgical Generator, FORCE 1C™, FORCE 2™ Generator, SurgiStat™ II or other envisioned generators which may perform different or enhanced functions. One such system is described in commonly-owned U.S. Pat. No. 6,033,399 entitled “ELECTROSURGICAL GENERATOR WITH ADAPTIVE POWER CONTROL”. Other systems have been described in commonly-owned U.S. Pat. No. 6,187,003 entitled “BIPOLAR ELECTROSURGICAL INSTRUMENT FOR SEALING VESSELS”.

In one embodiment, the generator 200 includes various safety and performance features including isolated output and independent activation of accessories. It is envisioned that the electrosurgical generator includes Valleylab's Instant Response™ technology features which provides an advanced feedback system to sense changes in tissue 200 times per second and adjust voltage and current to maintain appropriate power.

Electrosurgical instrument 10 can be any type of electrosurgical apparatus known in the available art, including but not limited to electrosurgical apparatuses that can grasp and/or perform any of the above mentioned electrosurgical procedures. One type of electrosurgical apparatus 10 may include bipolar forceps as disclosed in commonly-owned United States Patent Publication No. 2007/0173814 entitled “Vessel Sealer and Divider For Large Tissue Structures”, which is hereby incorporated by reference in its entirety for all purposes herein.

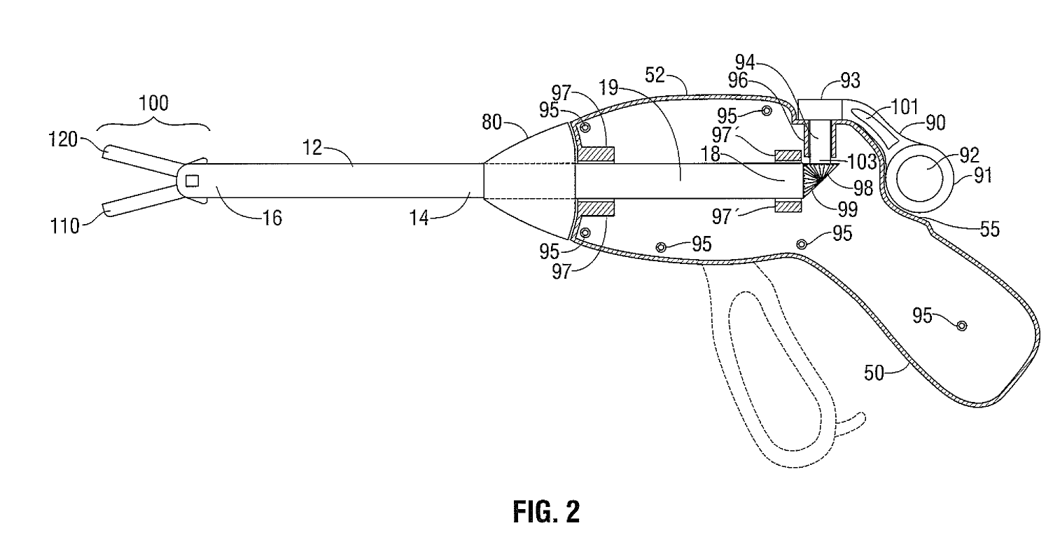

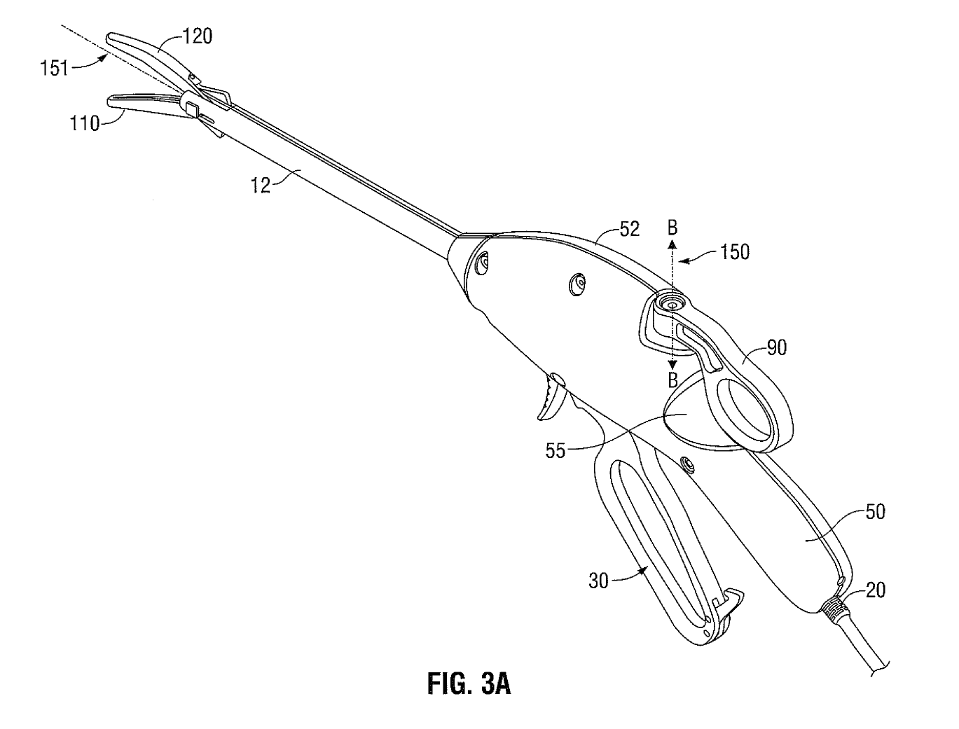

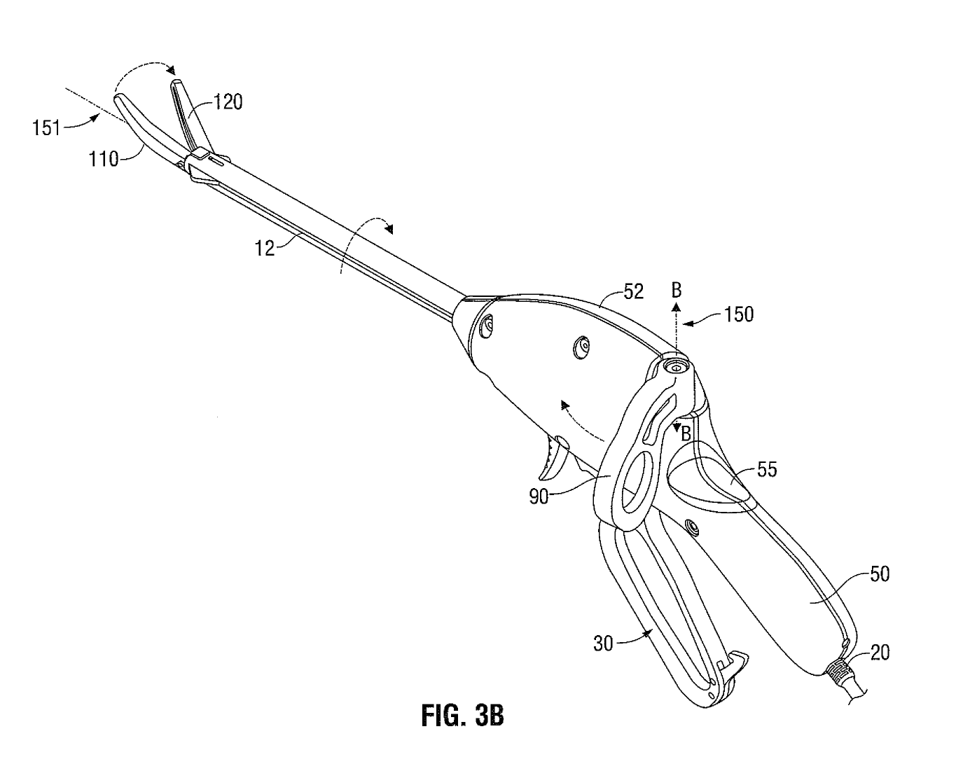

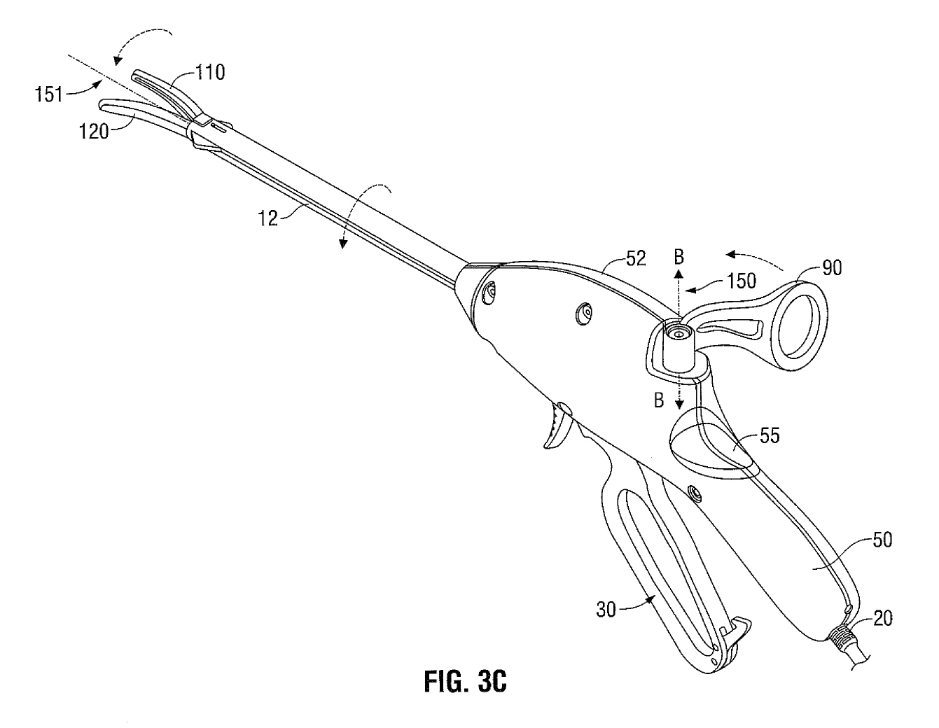

With reference now to FIGS. 1 and 2, electrosurgical instrument 10 is shown for use with various electrosurgical procedures and includes a housing 52 having a fixed handle assembly 50, a movable handle assembly 30, a trigger assembly 70, a rotating collar 80, a thumb lever assembly 90, a shaft 12, a jaw drive assembly (not explicitly shown), a shaft drive assembly 102, and an end effector 100, which mutually cooperate to grasp, seal and divide large tubular vessels and large vascular tissues. Although the majority of the figure drawings depict an electrosurgical instrument 10 for use in connection with endoscopic surgical procedures, the present disclosure may be used for more traditional open surgical procedures. Shaft 12 includes a distal end 16 dimensioned to mechanically engage the end effector 100, a central portion 14 which mechanically engages the housing 20, and a proximal end 18 dimensioned to engage lever drive assembly 102 as further described hereinbelow. Shaft 12 is rotatable approximately 180 degrees about the longitudinal axis A-A thereof and is operatively associated with housing 52.

Fixed handle assembly 50 may be integrally associated with housing 52. Movable handle 30 is movable relative to fixed handle 50. Fixed handle 50 may be oriented about 30 degrees relative to the longitudinal axis of shaft 12. Fixed handle 50 and/or movable handle 30 may include one or more ergonomic enhancing elements to facilitate handling, e.g., scallops, protuberances, elastomeric material, etc. In embodiments, movable handle 30 has an opening 40 defined therein which may facilitate grasping by permitting the fingers of a user to pass therethrough, as can be appreciated.

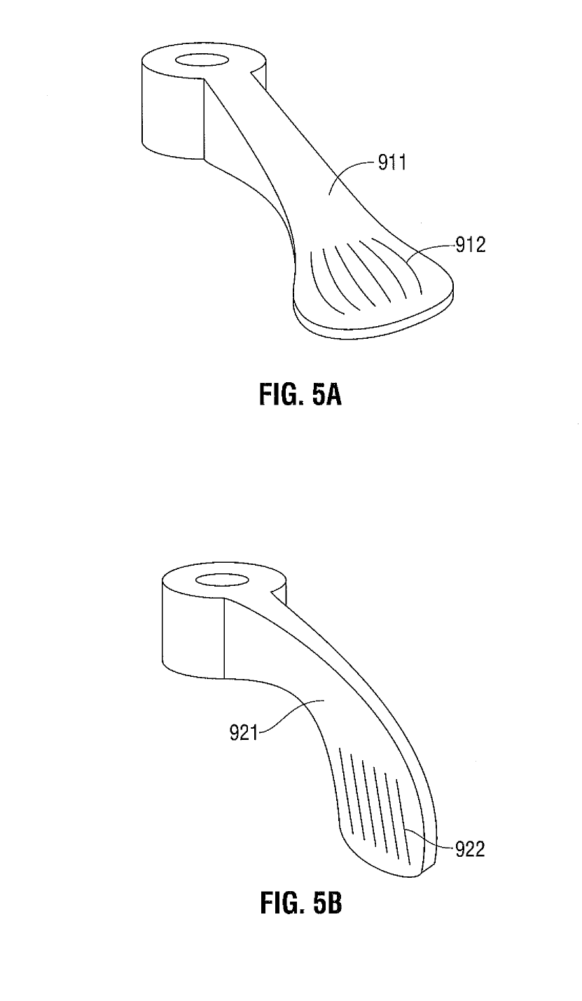

Thumb lever 90 is operatively associated with housing 52 and is rotatable through an arc of about 180 degrees about the rotational axis “B-B” (See FIG. 1). Thumb lever assembly 90, shaft 12, and lever drive assembly 102 cooperate to translate the side-to-side motion of thumb lever 90 about the B-B axis thereof into rotational motion of shaft 12 about the A-A axis. Thumb lever 90 includes a hub 93 disposed at the distal end of thumb lever 90 having a driveshaft 94 extending therefrom into housing 52 along axis “B-B”. Driveshaft 94 may be coupled to hub 93 by any suitable means, for example without limitation, by threaded fastener (not explicitly shown), adhesive, or clip. In embodiments, driveshaft 94 may be integrally formed with hub 93 and/or thumb lever 90. Thumb lever 90 may include a recess 101 configured to improve the rigidity and reduce the material volume thereof. Thumb lever 90 at a proximal end thereof includes a thumb ring 91 defining an opening 92 adapted to accommodate a finger, i.e., thumb, of a user. In other envisioned embodiments best illustrated by FIGS. 5A and 5B, thumb lever 90 at the proximal end may include a thumb saddle 911 or a thumb paddle 921. Thumb saddle 911 or thumb paddle 921 may additionally include texturing, protrusions, or ribbing 912, 922. In embodiments, housing 52 includes a contoured region 55 configured to provide clearance for thumb lever 90 and/or thumb ring 91.

Driveshaft 94 is supported within housing 52 by driveshaft sleeve 96 which may be integrally formed with housing 52. Driveshaft sleeve 96 has an inside diameter dimensioned to allow free rotation of driveshaft 94 within driveshaft sleeve 96 while maintaining alignment of driveshaft 94 with lever drive assembly 102. In embodiments, driveshaft sleeve 96 may include a bearing (not explicitly shown) such as without limitation ball bearing, roller bearing or friction bearing. In embodiments, the clearance between driveshaft 94 and driveshaft sleeve 96 may be dimensioned to achieve a predetermined amount of friction.

Lever drive assembly 102 includes bevel gear 98 that is disposed upon the lower end 103 of driveshaft 94 and bevel gear 99 that is disposed upon the proximal end 18 of shaft 12. Bevel gear 99 engages bevel gear 98 to communicate the side-to-side motion of thumb lever 90 about the B-B (i.e., vertical) axis thereof into rotational motion of shaft 12 about the A-A (i.e., longitudinal) axis. In embodiments, bevel gears 98 and 99 have a unity (1:1) gear ratio. In other envisioned embodiments, bevel gears 98 and 99 have a non-unity gear ratio whereby shaft 12 is driven at a rotational rate greater, or alternatively, less than, that of driveshaft 94. Bevel gears 98 and 99 may be arranged such that a clockwise rotation of driveshaft 94 imparts a clockwise rotation to shaft 12, or alternatively, a clockwise rotation of driveshaft 94 imparts a counterclockwise rotation to shaft 12. In yet other embodiments, the relationship between the rotation of driveshaft 94 and the rotation of shaft 12 is switchably selectable.

The present disclosure is not limited to the use of bevel gears to translate motion between the driveshaft and shaft. Other envisioned embodiments are disclosed wherein friction rollers, pulley and belts configurations, sprocket and chain configurations, and the like perform the function of lever drive assembly 102 and/or bevel gears 98 and 99.

Shaft 12 includes a shaft proximal portion 19 thereof that extends into housing 52. Shaft proximal portion 19 is supported within housing 52 by shaft sleeve 97 and 97′ which may be integrally formed with housing 52. Shaft sleeve 97, 97′ have an inside diameter dimensioned to allow free rotation of shaft 12 and thus shaft proximal portion 19 within sleeves 97, 97′ while maintaining alignment of shaft 12 and thus shaft proximal portion 19 with lever drive assembly 102, i.e., maintaining the engagement of bevel gears 98 and 99. In embodiments, shaft sleeves 97, 97′ may include a bearing (not explicitly shown) such as without limitation ball bearing, roller bearing or friction bearing. In embodiments, the clearance between shaft proximal portion 19 and driveshaft sleeves 97, 97′ may be dimensioned to achieve a predetermined amount of friction.

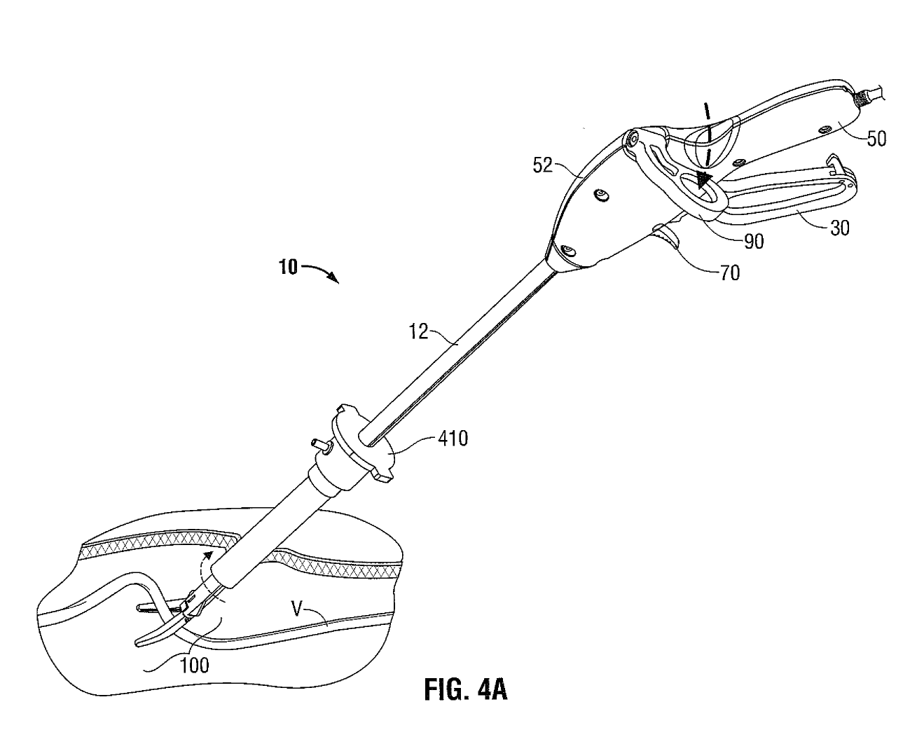

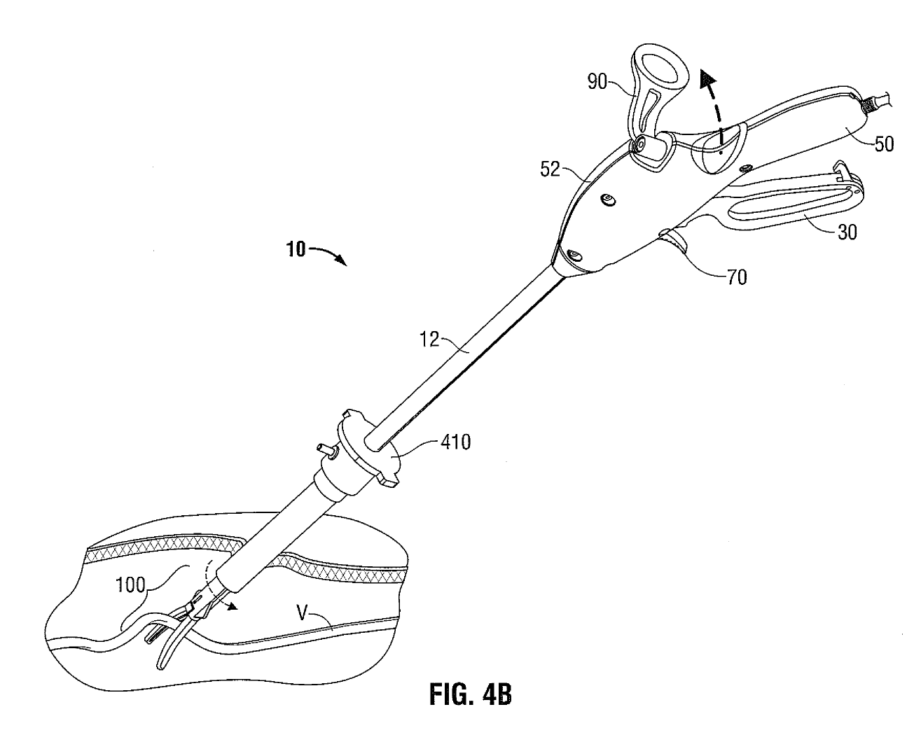

In use, electrosurgical instrument 10 may be introduced to the surgical site of patient P through a cannula or trocar port 410 as best illustrated in FIG. 4A whereby end effector 100 may be positioned to grasp and/or seal vessel V. As can be seen, thumb lever 90 has been moved into a left position which, in the illustrated embodiment, has caused end effector 100 to advantageously rotate to a clockwise position that is well-suited for grasping vessel V, while permitting housing 52 to remain in substantially fixed position that may remain well-placed, for example, in the hand of the surgeon. Turning now to FIG. 4B, electrosurgical instrument 10 is positioned to grasp vessel V′ of patient P′, where vessel V′ follows a substantially different path from that of vessel V of FIG. 4A. Thumb lever 90 has accordingly been moved into a right position to cause end effector 100 to rotate to a counterclockwise position well-suited for grasping vessel V′, while permitting housing 52 to remain in substantially fixed position as described hereinabove.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical instrument, comprising:
a housing having a movable thumb handle rotatable about a first axis;
a driveshaft having a first end and a second end, the driveshaft coupled at a first end thereof to the thumb handle and longitudinally rotatable about the first axis;
a shaft coupled to the housing, the shaft rotatable about a second axis defined longitudinally therethrough and having an end effector disposed at a distal end thereof; and
a drive assembly configured to couple a second end of the driveshaft and a proximal end of the rotatable shaft, wherein a rotation of the driveshaft is translated into a rotation of the rotatable shaft.

2. The electrosurgical instrument in accordance with claim 1, wherein the drive assembly comprises:
a driving element operably coupled to a second end of the driveshaft;
a driven element operably coupled to a proximal end of the shaft;
wherein the driving element and driven element cooperate to effect rotation of the shaft.

3. The electrosurgical instrument in accordance with claim 2, wherein the driving element is selected from the group consisting of a bevel gear, a friction roller, a pulley, and a sprocket.

4. The electrosurgical instrument in accordance with claim 2, wherein the driven element is selected from the group consisting of a bevel gear, a friction roller, a pulley, and a sprocket.

5. The electrosurgical instrument in accordance with claim 1, wherein rotation of the driveshaft imparts a similar rotation to the shaft.

6. The electrosurgical instrument in accordance with claim 1, wherein a rotation of the driveshaft imparts an opposite rotation to the shaft.

7. The electrosurgical instrument in accordance with claim 1, wherein the proximal end of the thumb lever includes a thumb ring.

8. The electrosurgical instrument in accordance with claim 1, wherein the proximal end of the thumb lever includes a thumb paddle.

9. The electrosurgical instrument in accordance with claim 1, wherein the proximal end of the thumb lever includes a thumb saddle.

10. The electrosurgical instrument in accordance with claim 1, wherein the end effector assembly includes two jaw members movable from a first position in spaced relation relative to one another to at least a second position closer to one another for grasping tissue therebetween, each of the jaw members adapted to connect to an electrosurgical energy source such that the jaw members are capable of conducting energy through tissue held therebetween.

11. An electrosurgical system, comprising:
an electrosurgical module configured to generate electrosurgical energy;
an electrosurgical instrument comprising:
a housing having a movable thumb handle rotatable about a first axis;
a driveshaft having a first end and a second end, the driveshaft coupled at a first end thereof to the thumb handle and longitudinally rotatable about the first axis;
a shaft coupled to the housing, the shaft rotatable about a second axis defined longitudinally therethrough and having an end effector disposed at a distal end thereof, wherein the end effector is configured to deliver electrosurgical energy to tissue; and
a drive assembly configured to couple a second end of the driveshaft and a proximal end of the rotatable shaft, wherein a rotation of the driveshaft is translated into a rotation of the rotatable shaft; and
a cable assembly configured to operably couple the electrosurgical module and the electrosurgical instrument.

12. The electrosurgical system in accordance with claim 11, wherein the drive assembly comprises:
a driving element operably coupled to a second end of the driveshaft;
a driven element operably coupled to a proximal end of the shaft;
wherein the driving element and driven element cooperate to effect rotation of the shaft.

13. The electrosurgical system in accordance with claim 12, wherein the driving element is selected from the group consisting of a bevel gear, a friction roller, a pulley, and a sprocket.

14. The electrosurgical system in accordance with claim 12, wherein the driven element is selected from the group consisting of a bevel gear, a friction roller, a pulley, and a sprocket.

15. The electrosurgical system in accordance with claim 11, wherein a rotation of the driveshaft imparts a similar rotation to the shaft.

16. The electrosurgical system in accordance with claim 11, wherein a rotation of the driveshaft imparts an opposite rotation to the shaft.

17. A method of performing electrosurgery, comprising the steps of:
providing an electrosurgical module configured to generate electrosurgical energy;
providing an electrosurgical instrument comprising:
a housing having a movable thumb handle rotatable about a first axis;
a driveshaft having a first end and a second end, the driveshaft coupled at a first end thereof to the thumb handle and longitudinally rotatable about the first axis;
a shaft coupled to the housing, the shaft rotatable about a second axis defined longitudinally therethrough and having an end effector disposed at a distal end thereof, wherein the end effector is configured to deliver electrosurgical energy to tissue; and
a drive assembly configured to couple a second end of the driveshaft and a proximal end of the rotatable shaft, wherein a rotation of the driveshaft is translated into a rotation of the rotatable shaft;
providing a cable assembly configured to operably couple the electrosurgical module and the electrosurgical instrument;
actuating the movable thumb handle to rotate the end effector; and
applying electrosurgical energy to tissue.

18. The method in accordance with claim 17, wherein the end effector assembly provides two jaw members movable from a first position in spaced relation relative to one another to at least a second position closer to one another for grasping tissue therebetween.

19. The method in accordance with claim 18, further comprising the step of:
positioning the jaw members around tissue therebetween;
moving the jaw members from a first position in spaced relation relative to one another to at least a second position closer to one another to seal tissue therebetween.
